(19) United States
(12) Patent Application Publication
(10) Pub. No.: US 2026/0345549 A1
(43) Pub. Date: Nov. 5, 2026

(54) UROLITHIN FOR IMPROVING PERFORMANCE IN ELITE AND SUB-ELITE ATHLETES

(71) Applicant: Amazentis SA, Lausanne (CH)

(72) Inventors: Christopher L. Rinsch, Lausanne (CH); Anurag Singh, Lausanne (CH)

(21) Appl. No.: 19/488,215

(22) PCT Filed: May 28, 2024
(86) PCT No.: PCT/IB2024/000262

§ 371 Date: Nov. 26, 2025

Related U.S. Application Data
(60) Provisional application No. 63/469,215, filed on May 26, 2023.

(51) Int. Cl. A61K 31/366; A61P 21/00

(52) U.S. Cl. CPC A61K 31/366; A61P 21/00

(57) ABSTRACT

Disclosed are methods of using Urolithin A to enhance physical performance in elite athletes and sub-elite athletes.

### RELATED APPLICATION

This application claims the benefit of priority to U.S. Provisional Patent Application No. 63/469,215, filed May 26, 2023.

### BACKGROUND

Urolithins have been shown to have been shown to be highly biologically active metabolites which can affect a number of health conditions. In particular, Urolithin A shows promise as a means of improving muscle function in humans.

In training for and participating in athletic competitions, athletes experience impairment in muscle recovery and inflammation which can hinder peak performance. Even the slightest improvements in muscle function, post-exercise recovery, and endurance can yield dramatic results in competition. Urolithin A (UA) has shown promise in improving muscle function in subjects with some degree of mitochondrial dysfunction (e.g., elderly or overweight populations); however, relatively little is known about the effects of UA on muscle function and performance in elite and/or sub-elite athletes.

### SUMMARY OF THE INVENTION

In certain aspects, the present disclosure relates to methods of enhancing physical performance, comprising administering to a subject in need thereof an effective amount of Urolithin A, wherein the subject is an elite athlete or sub-elite athlete.

In further aspects, the present disclosure relates to methods of enhancing physical recovery, comprising administering to a subject in need thereof an effective amount of Urolithin A, wherein the subject is an elite athlete or sub-elite athlete.

In yet further aspects, the present disclosure relates to methods of enhancing physical endurance, comprising administering to a subject in need thereof an effective amount of Urolithin A, wherein the subject is an elite athlete or sub-elite athlete.

### BRIEF DESCRIPTION OF THE DRAWINGS

FIG. 1 is a chart depicting the study design in Example 1.

FIG. 2 is a chart depicting study demographics.

FIG. 3 is a chart depicting the results in 3 km running performance times in elite athletes involved in the study.

FIG. 4 is a chart depicting the change in 3 km running performance times in elite athletes involved in the study.

FIG. 5 is a chart depicting the reported post-race exertion (RPE) in the group supplemented with 1000 mg Urolithin A (UA).

FIG. 6 is a chart depicting the reported post-race exertion (RPE) in the group supplemented with a placebo.

FIG. 7 is a chart depicting the change in RPE reported by the UA and placebo arms of the study.

FIG. 8 is a chart depicting measured maximal oxygen consumption (VO₂ₘₐₓ) in the Urolithin A and Placebo arms.

FIG. 9 is a chart depicting the change in measured maximal oxygen consumption (VO₂ₘₐₓ) in the Urolithin A and Placebo arms.

FIG. 10 is a chart depicting the effects of UA supplementation on creatine kinase (CK) concentration.

FIG. 11 is a chart depicting the effects of placebo supplementation on CK concentration.

FIG. 12 is a chart depicting the effects of UA supplementation on CK concentration during training (after downhill run).

FIG. 13 is a chart depicting the effects of placebo supplementation on CK concentration during training (after downhill run).

FIG. 14 is a chart depicting the effects of UA supplementation on C-reactive protein (CRP) concentration during training (after downhill run).

FIG. 15 is a chart depicting the effects of placebo supplementation on CRP concentration during training (after downhill run).

FIG. 16A is a dot plot, showing the three GO CCs commonly activated in both UA and Placebo comparisons. GO CCs are sorted by the UA protein ratio on the x-axis. Dot size represents the statistical significance of the enrichment (Benjamini-Hochberg-adjusted p-value).

FIG. 16B is a Venn diagram (top panel) showing the overlap of significantly activated pathways (Gene Ontology Cellular Components, “GO CCs”) between Urolithin A (UA) and Placebo across experimental visits (from baseline to post-treatment). See the Materials and Methods section for more details. Bottom panel: Identical analysis as in the top panel, but depicting significantly repressed pathways.

FIG. 16C is a dot plot depicting the top repressed and activated GO CCs in the UA comparison, adjusted for Placebo effects (see Materials and Methods for adjustment details). GO CCs are ordered by Protein ratio. Dot size reflects the Protein Ratio, and color denotes the statistical significance of the enrichment (Benjamini-Hochberg-adjusted p-value). There leftmost dots are placebo; three rightmost dots are UA.

FIG. 16D are Paired boxplots illustrating the normalized protein expression for the top-6 core enrichment proteins within the mitochondrial protein-containing complex GO CC term (see (c)). Each plot compares changes between baseline and post-treatment visits for both Placebo and UA groups, with lines connecting individual subjects' expression values across visits.

### DETAILED DESCRIPTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

“Urolithin A” is 3,8-dihydroxy-6H-dibenzo[b,d]pyran-6-one, which is represented by the following structure:

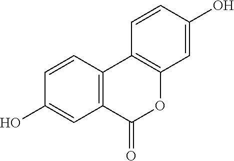 [SMILES: O=c1oc2cc(O)ccc2c2ccc(O)cc12]

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

“Optional” or “optionally” means that the subsequently described event or circumstances may or may not occur, and that the description includes instances where said event or circumstance occurs and instances in which it does not. For example, “optionally substituted aryl” means that the aryl radical may or may not be substituted, and that the description includes both substituted aryl radicals and aryl radicals having no substitution.

As used herein, the term “treat” as used in connection with a disease, disorder, or condition of a subject, means to reduce by a detectable amount at least one clinical or objective manifestation of the disease, disorder, or condition of a subject. In one embodiment, the term “treat” used in connection with a disease, disorder, or condition of a subject, means to cure the disease, disorder, or condition of a subject.

As used herein, a “food product” refers to a product prepared from a natural food. Non-limiting examples of food products include juices, wines, concentrates, jams, jellies, preserves, pastes, and extracts.

As used herein, a “nutritional supplement” refers to a product suitable for consumption or other administration principally for its health-promoting properties rather than its caloric content.

As used herein, the term “maximal oxygen consumption” (VO₂ₘₐₓ) refers to the maximum amount of oxygen that an individual can utilize during intense or maximal exercise, as measured, e.g., by comparing the volume and gas concentrations of inspired and expired air.

As used herein, the term “elite” athlete may refer to a human subject who has a VO₂ₘₐₓ of greater than 65 mL Kg⁻¹ min⁻¹, a 3 km running personal best time below 9 minutes, or both.

As used herein, the term “sub-elite” athlete may refer to a human subject who has a VO₂ₘₐₓ from 60 mL Kg⁻¹ min⁻¹ to 65 mL Kg⁻¹ min⁻¹, a 3 km running personal best time from 9 to 10 minutes, or both.

### Methods of Enhancing Physical Performance

In certain aspects, the present disclosure relates to methods of enhancing physical performance in a subject in need thereof, comprising administering to the subject an effective amount of Urolithin A, and the subject is an elite athlete or sub-elite athlete.

In certain embodiments, enhancing physical performance comprises at least one effect selected from the group consisting of enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In further embodiments, enhancing physical performance comprises enhancing muscle performance during a high-intensity aerobic activity. In yet further embodiments, enhancing physical performance comprises increasing aerobic endurance during a high-intensity aerobic activity. In still further embodiments, enhancing physical performance comprises increasing resting metabolic rate (RMR). In certain embodiments, enhancing physical performance results in an improvement in athletic performance. In further embodiments, enhancing physical performance results in an improvement in footrace completion times. In yet further embodiments, enhancing physical performance results in a decrease in Ratings of Perceived Exertion (RPE).

### Methods of Enhancing Physical Recovery

In further aspects, the present disclosure relates to methods of enhancing physical recovery, comprising administering to a subject in need thereof an effective amount of Urolithin A, wherein the subject is an elite athlete or sub-elite athlete.

In certain embodiments, physical recovery is enhanced after a high-intensity aerobic activity. In further embodiments, enhancing physical recovery comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In yet further embodiments, enhancing physical recovery comprises enhancing muscle recovery after a high-intensity aerobic activity. In still further embodiments, enhancing physical recovery comprises reducing muscle soreness after a high-intensity aerobic activity. In certain embodiments, enhancing physical recovery comprises lowering creatine kinase (CK) levels in the subject, compared to baseline, following an aerobic activity as measured by area under the plasma concentration-time curve of CK (AUC_{CK}). In further embodiments, enhancing recovery comprises lowering C-reactive protein (CRP) levels in the subject, compared to baseline, following an aerobic activity as measured by area under the plasma concentration-time curve (AUC_{CRP}).

### Methods of Enhancing Physical Endurance

In yet further aspects, the present disclosure relates to methods of enhancing endurance comprising administering to a subject in need thereof an effective amount of Urolithin A, wherein the subject is an elite athlete or sub-elite athlete.

In certain embodiments, the method comprises enhancing physical endurance during a high-intensity aerobic activity. In further embodiments, enhancing physical endurance comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In yet further embodiments, enhancing physical endurance comprises enhancing muscle endurance. In still further embodiments, enhancing physical endurance comprises increasing maximal oxygen consumption (VO₂ₘₐₓ) in the subject.

In certain embodiments, methods of the present disclosure comprise administering to a subject in need thereof an effective amount of Urolithin A, wherein the subject is an elite athlete or sub-elite athlete. In some such embodiments, the subject is an elite athlete. In certain embodiments, the subject has a VO₂ₘₐₓ of greater than 65 mL kg-1 min-1. In further embodiments, the subject has a 3 km running personal best time below 9 minutes. In other embodiments, the subject is a sub-elite athlete. In certain such embodiments, the subject has a VO₂ₘₐₓ of from about 60 mL kg-1 min-1 to about 65 mL kg-1 min-1. In further embodiments, the subject has a 3 km running personal best time from about 9 minutes to about 10 minutes. In certain embodiments, the age of the subject is from about 18 years to about 45 years.

In certain embodiments, methods of the disclosure comprising administering to a subject in need thereof an effective amount of Urolithin A. In some such embodiments, the effective amount of Urolithin A is from about 70 mg/day to about 1050 mg/day. In further embodiments, the effective amount of Urolithin A is about 500 mg/day. In yet further embodiments, the effective amount of Urolithin A is about 1000 mg/day. In certain embodiments, the effective amount of Urolithin A is administered once daily. In further embodiments, the effective amount of Urolithin A is administered once daily for a period of at least about 28 days. In yet further embodiments, the effective amount of Urolithin A is administered once daily for a training period. In still further embodiments, the training period is from about 28 days to about 168 days, from about 28 days to about 140 days, from about 28 days to about 112 days, from about 28 days to about 84 days, or from about 28 days to about 56 days. In certain embodiments, the training period is about 28 days.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The invention now being generally described, it will be more readily understood by reference to the following, which is included merely for purposes of illustration of certain aspects and embodiments of the present invention, and is not intended to limit the invention.

### Example 1: Clinical Study of Urolithin a Effects on Elite and Sub-Elite Athlete Performance

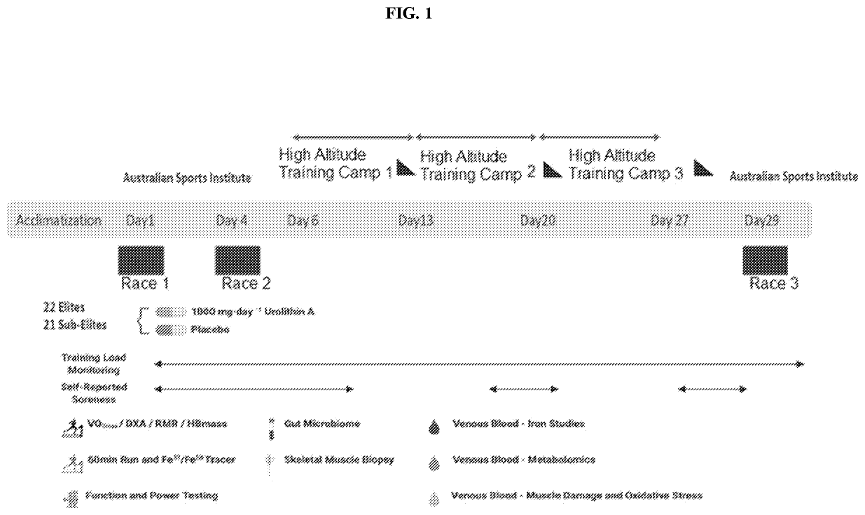

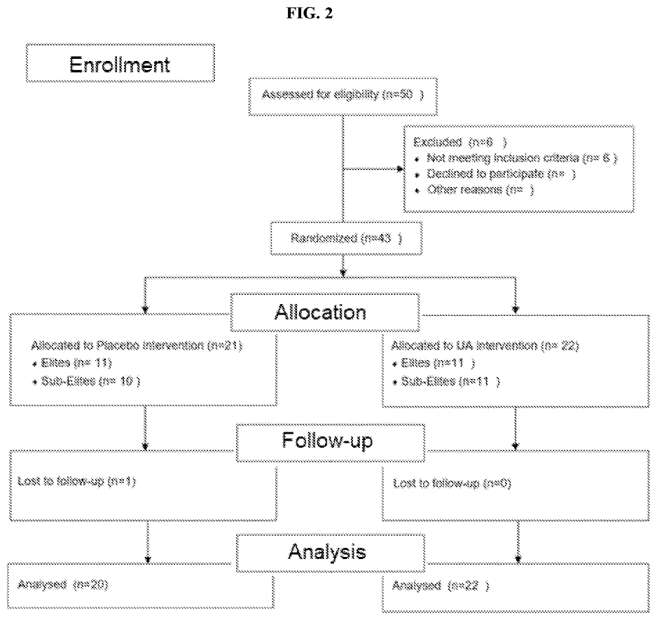

A clinical study was carried out to investigate effects of Urolithin A (UA) on performance, recovery, and endurance in elite and sub-elite athletes (FIG. 1). Exercise and training adaptation was continuous in both treatment arms. Physiological outcomes measured in the study include: performance in a 3 km track race, aerobic endurance, resting metabolic rate, and body composition (e.g., BMI). Recovery outcomes measured in the study include: physiological concentrations of recovery biomarkers creatine kinase (CK) and C-reactive protein (CRP) as measured by area under the plasma drug concentration-time curve (AUC), delayed-onset muscle soreness, mitochondrial health, and impact on iron absorption as measured by blood iron concentrations. Participants' ages ranged from 18-45 years. Participants ran >70 km per week. Participants in the “elite” participants were required to have a 3000 m running personal best time below 09:00 (mm: ss), and/or a VO₂ₘₐₓ result greater than 65 ml·kg⁻¹·min⁻¹. Participants in the “sub-elite” cohort were required to have a 3000 m running personal best faster than 10:00 min and/or a VO₂ₘₐₓ greater than 60 ml·kg⁻¹·min⁻¹, but which did not qualify for the elite cohort. Subjects were randomized based on BMI, running time, and baseline VO₂ₘₐₓ. Study demographics are overviewed in Table 1 and FIG. 2.

TABLE 1
Summary of study demographics

| | UA inter- | Placebo |
|---|---|---|
| | vention arm | arm |
| n(Elite participants) | 11 | 11 |
| n(Sub-elite participants) | 11 | 10 |
| Mean age (yrs) | 28.9 | 26 |
| Mean BMI (kg m⁻²) | 20.6 | 20.5 |
| Mean VO₂ₘₐₓ | 66.8 | 66.1 |
| (mL kg⁻¹ min⁻¹) | | |
| Drop-outs | 0 | 1 |
| Total participants analyzed | 22 | 20 |

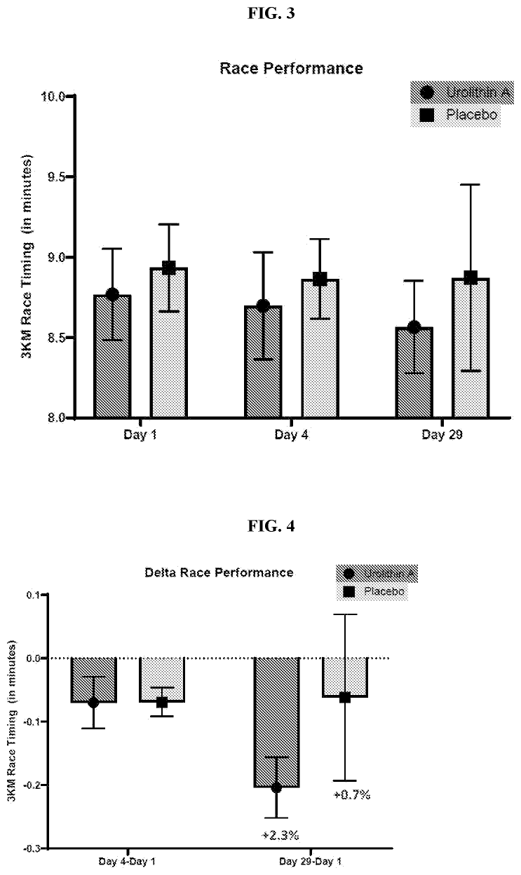

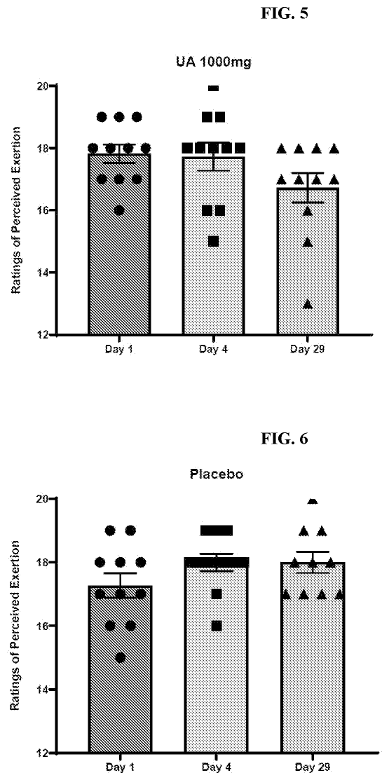

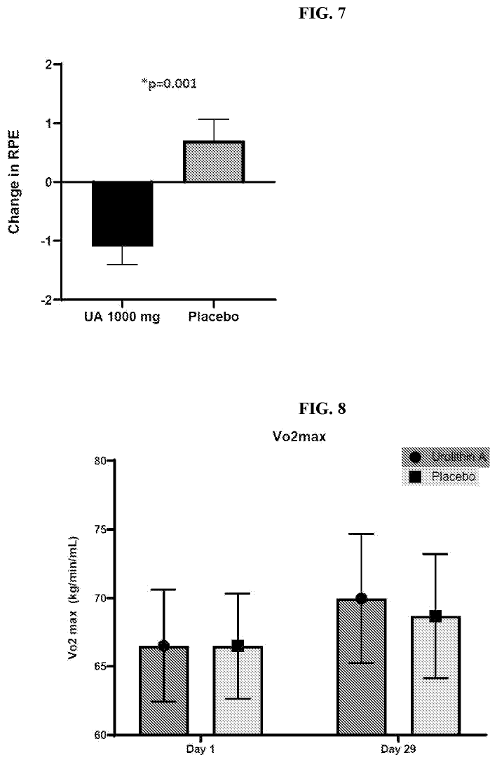

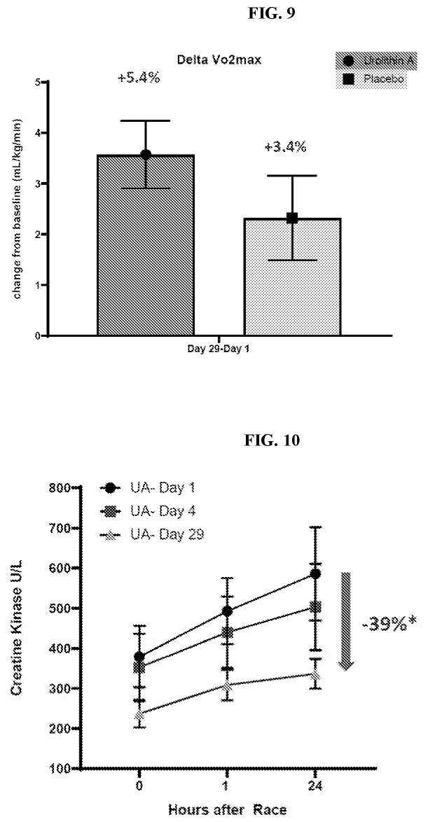

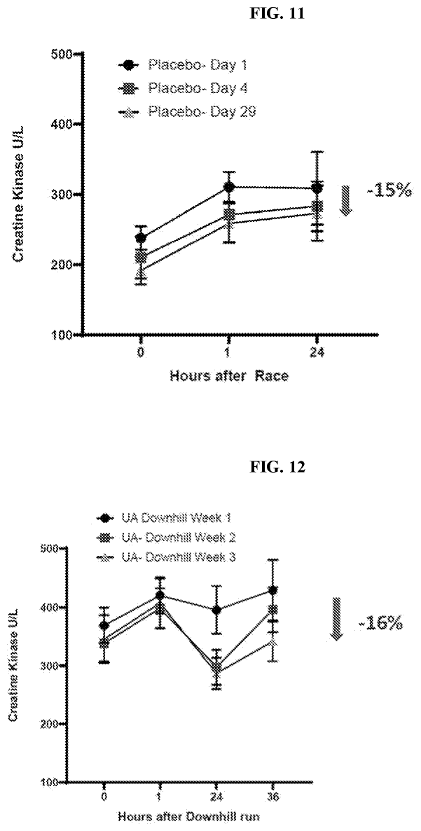

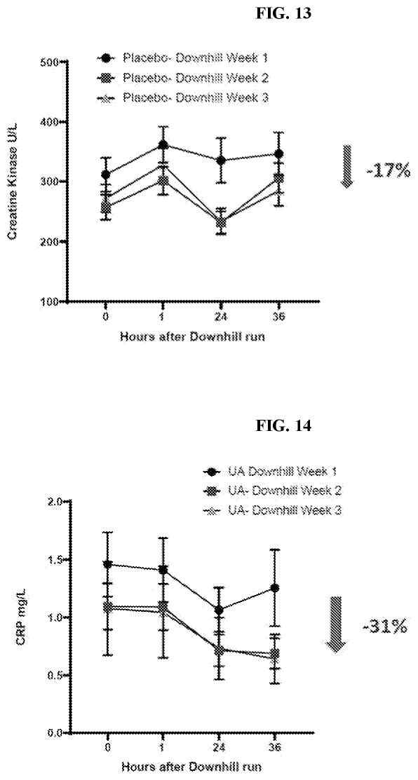

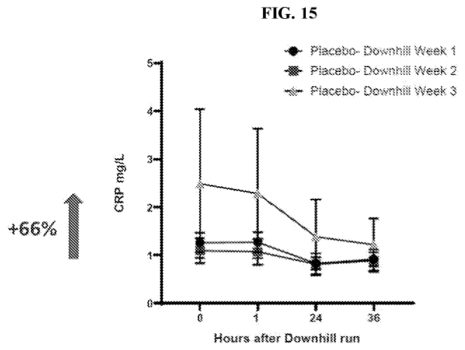

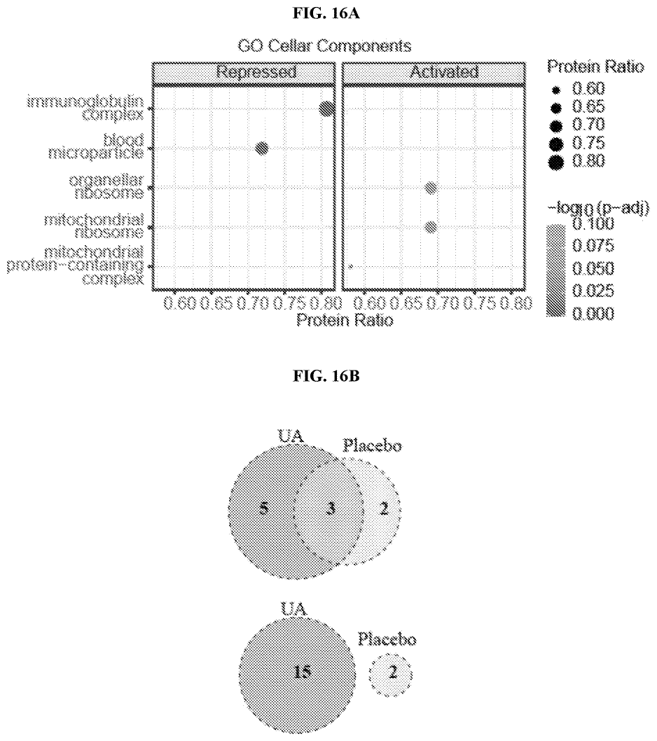

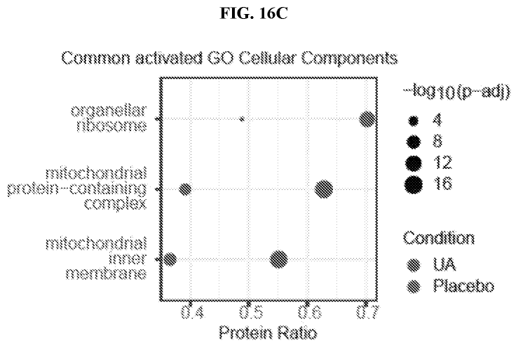

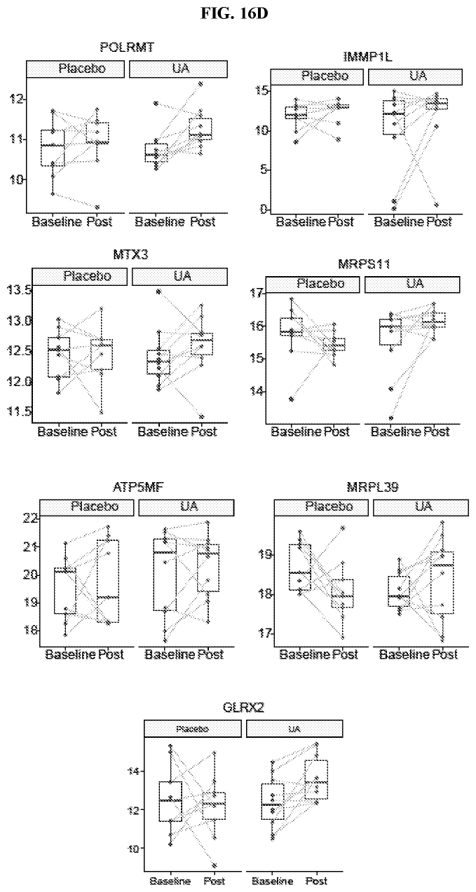

The results of the clinical study are summarized in Table 2, and FIGS. 3-15. In summary, the Urolithin A (+exercise) group demonstrated meaningful (but not statistically significant) improvements in race performance (3 km race) and endurance (VO₂ₘₐₓ), compared to placebo (+exercise) in elite athletes. Elite athletes perceived significantly less exertion (RPE) after 4 weeks supplementation (after 3^{rd} race 3 km sprint) in Urolithin A group compared to placebo. Elite athletes on Urolithin A supplementation also had significantly less muscle damage after 4 weeks of supplementation (as measured by AUC_{CK} levels; after final 3^{rd} race compared to placebo). Urolithin A supplementation had a significant impact on inflammation (measured by AUC_{CRP}) in elite and sub-elite athletes at the end of the last high-altitude training camp (day 26).

TABLE 2
Summary of results

| | UA treatment group | Placebo |
|---|---|---|
| Endpoints | (1000 mg UA + Exercise)) | (+Exercise only) |
| 3 km Race Performance | +2.3% | +0.7% |
| (Day 29 compared to | (20 seconds | (6.2 seconds |
| baseline) | improvement) | improvement) |
| RPE | −6.2%* | +3.9% |
| (Ratings of Perceived | (1.1-point | (0.7 point |
| Exertion)- (Day 29 | scale lower) | higher) |
| compared to baseline) | | |
| CK | −39%* lower | −15.8% lower |
| (Post-3rdRace Day 29) | (AUC) | (AUC) |
| CK | −16% (AUC) | −17% (AUC) |
| (Post-3rdTraining | | |
| Camp Day 26) | | |
| CRP | −31%* (AUC) | +66% (AUC) |
| (Post-3rdTraining | | |
| Camp Day 26) | | |
| Vo2 max | +5.4% | +3.4% |
| (Day 29 compared to | (3.57 kg/ml/min) | (2.32 kg/ml/min) |
| baseline) | improvement | improvement |

Data on performance endpoints and recovery during performance is collected in elite participants only (n = 11/arm).
*significant from baseline compared to placebo group.

### Example 2: Differential Protein Expression Analysis

To assess protein expression changes between baseline and post-UA treatment visits while accounting for Placebo effects, a robust linear mixed model framework was employed, and in particular the msqrob2 package (Sticker et al. Molecular & Cellular Proteomics 19.7 (2020): 1209-1219). Raw protein intensity values underwent log transformation followed by normalization using the median-center method. The model was formulated as follows: treatment+visit+treatment: visit, where treatment represents either UA or placebo, and visit indicates baseline or post-treatment visit. Visit was treated as an interaction term in the model. To assess the mean log 2 expression between post-UA and baseline, corrected for Placebo effects, the contrast “(Intercept)+post+UA:post” was employed, with the respective statistical test being “post+UA:post=0”.

### Example 3: Gene Set Enrichment Analysis (GSEA)

For GSEA enrichment, the Cellular Components collection of the Gene Ontology database (GO CCs) was utilized. Enrichment analysis was performed separately for the “simple” and “robust regression” models using the R package ClusterProfiler (Yu, G. et al. OMICS: A Journal of Integrative Biology 2012, 16 (5): 284-287). Log 2 fold change values between visits served as the protein ranking metric. Subsequently, the enrichment of GO CC gene sets among up- or down-regulated proteins was statistically tested. The minimum and maximum gene set sizes were set to 10 and 500, respectively. Adjusted p-values were determined using the BH method (Benjamini, Y. and Hochberg, Y. Journal of the Royal Statistical Society: Series B (Methodological), 57(1): 289-300). Terms with an adjusted p-value≤0.05 were considered statistically significant and further characterized as activated (NES>0) or repressed (NES<0) based on the respective Normalized Enrichment Score (NES) sign. Visualizations included Venn diagrams (Chen, H. VennDiagram: Generate High-Resolution Venn and Euler Plots. R package version 1.7.3) to depict common and unique activated/repressed GSEA GO CCs between Placebo and UA comparisons, dot plots of Protein Ratio, and paired boxplots of normalized protein expression values, highlighting top enrichment proteins within specific terms, specifically the “mitochondrial protein-containing complex” GO CC term in the “robust regression” analysis.

### INCORPORATION BY REFERENCE

All patents and published patent applications mentioned in the description above are incorporated by reference herein in their entirety.

### EQUIVALENTS

Having now described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

We claim:

1. A method of enhancing physical performance, comprising administering to a subject in need thereof an effective amount of Urolithin A, wherein the subject is an elite athlete or sub-elite athlete.

2. The method of claim 1, wherein enhancing physical performance comprises at least one effect selected from the group consisting of enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage.

3. The method of claim 1 or 2, wherein enhancing physical performance comprises enhancing muscle performance during a high-intensity aerobic activity.

4. The method of any one of claims 1-3, wherein enhancing physical performance comprises increasing aerobic endurance during a high-intensity aerobic activity.

5. The method of any one of claims 1-4, wherein enhancing physical performance comprises increasing resting metabolic rate (RMR).

6. The method of any one of claims 1-5, wherein enhancing physical performance results in an improvement in athletic performance.

7. The method of any one of claims 1-6, wherein enhancing physical performance results in an improvement in footrace completion times.

8. The method of any one of claims 1-7, wherein enhancing physical performance results in a decrease in Ratings of Perceived Exertion (RPE).

9. A method of enhancing physical recovery, comprising administering to a subject in need thereof an effective amount of Urolithin A, wherein the subject is an elite athlete or sub-elite athlete.

10. The method of claim 9, wherein physical recovery is enhanced after a high-intensity aerobic activity.

11. The method of claim 9, wherein enhancing physical recovery comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage.

12. The method of claim 11, wherein enhancing physical recovery comprises enhancing muscle recovery after a high-intensity aerobic activity.

13. The method of claim 12, wherein enhancing physical recovery comprises reducing muscle soreness after a high-intensity aerobic activity.

14. The method of any one of claims 9-13, wherein enhancing physical recovery comprises lowering creatine kinase (CK) levels in the subject, compared to baseline, following an aerobic activity as measured by area under the plasma concentration-time curve of CK (AUC_{CK}).

15. The method of any one of claims 9-14, wherein enhancing recovery comprises lowering C-reactive protein (CRP) levels in the subject, compared to baseline, following an aerobic activity as measured by area under the plasma concentration-time curve (AUC_{CRP}).

16. A method of enhancing physical endurance, comprising administering to a subject in need thereof an effective amount of Urolithin A, wherein the subject is an elite athlete or sub-elite athlete.

17. The method of claim 16, wherein the method comprises enhancing physical endurance during a high-intensity aerobic activity.

18. The method of claim 16 or 17, wherein enhancing physical endurance comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage.

19. The method of claim 18, wherein enhancing physical endurance comprises enhancing muscle endurance.

20. The method of any one of claims 16-19, wherein enhancing physical endurance comprises increasing maximal oxygen consumption (VO₂ₘₐₓ) in the subject.

21. The method of any one of claims 1-20, wherein the subject is an elite athlete.

22. The method of claim 21, wherein the subject has a VO₂ₘₐₓ of greater than 65 mL kg-1 min-1.

23. The method of claim 21 or 22, wherein the subject has a 3 km running personal best time below 9 minutes.

24. The method of any one of claims 1-20, wherein the subject is a sub-elite athlete.

25. The method of claim 24, wherein the subject has a VO₂ₘₐₓ of from about 60 mL kg-1 min-1 to about 65 mL kg-1 min-1.

26. The method of claim 24 or 25, wherein the subject has a 3 km running personal best time from about 9 minutes to about 10 minutes.

27. The method of any one of claims 1-26, wherein the effective amount of Urolithin A is from about 70 mg/day to about 1050 mg/day.

28. The method of any one of claims 1-27, wherein the effective amount of Urolithin A is about 500 mg/day.

29. The method of any one of claims 1-27, wherein the effective amount of Urolithin A is about 1000 mg/day.

30. The method of any one of claims 1-29, wherein the effective amount of Urolithin A is administered once daily.

31. The method of any one of claims 1-30, wherein the effective amount of Urolithin A is administered once daily for a period of at least about 28 days.

32. The method of any one of claims 1-30, wherein the effective amount of Urolithin A is administered once daily for a training period.

33. The method of claim 32, wherein the training period is from about 28 days to about 168 days.

34. The method of claim 32, wherein the training period is from about 28 days to about 140 days.

35. The method of claim 32, wherein the training period is from about 28 days to about 112 days.

36. The method of claim 32, wherein the training period is from about 28 days to about 84 days.

37. The method of claim 32, wherein the training period is from about 28 days to about 56 days.

38. The method of claim 32, wherein the training period is about 28 days.

39. The method of any one of claims 1-38, wherein the age of the subject is from about 18 years to about 45 years.
